(19) United States
(12) Patent Application Publication
(10) Pub. No.: US 2024/0210917 A1
(43) Pub. Date: Jun. 27, 2024

(54) METHOD AND SYSTEM FOR OPTIMIZING AN ALKANE DEHYDROGENATION OPERATION

(71) Applicant: PTT GLOBAL CHEMICAL PUBLIC COMPANY LIMITED, Chatuchak (TH)

(72) Inventors: Songphon CHATTHAMMANAT, Chatuchak (TH); Sitthiphong PENGPANICH, Chatuchak (TH); Kaew-arpha THAVORNPRASERT, Chatuchak (TH); Noppon LERTLUKKANASUK, Chatuchak (TH)

(73) Assignee: PTT GLOBAL CHEMICAL PUBLIC COMPANY LIMITED, Chatuchak (TH)

(21) Appl. No.: 18/088,665

(22) Filed: Dec. 26, 2022

(51) Int. Cl. G05B 19/4155; G06N 5/022

(52) U.S. Cl. CPC G05B 19/4155; G06N 5/022; C10G 9/00; G05B 2219/37583

(57) ABSTRACT

The present invention relates to a method for optimizing an alkane dehydrogenation operation. The method comprises the steps of obtaining historical data of a plurality of production parameter data, filtering abnormal data of the said historical data, developing a product yield prediction model and a coking rate prediction model, and processing the product yield prediction model and the coking rate prediction model, and determining optimum production parameter data based on the predicted product yield and the predicted coking rate. The present invention further relates to a system for optimizing an alkane dehydrogenation operation which comprises a production data detecting and storage unit and a processor configured to obtain historical data of the production parameter data, filter abnormal data of the historical data of the production parameter data, develop a product yield prediction model and a coking rate prediction model, and provide a process efficiency analysis model with a machine learning processing unit for processing the product yield prediction model and the coking rate prediction model to predict a product yield and a coking rate, and determine optimum production parameter data based on the predicted product yield and the predicted coking rate.

### TECHNICAL FIELD

The present invention relates to a method and system for optimizing an alkane dehydrogenation operation.

### BACKGROUND OF THE INVENTION

An alkane dehydrogenation process, in particular, a propane dehydrogenation process is a technology for producing propylene. The propylene can then be used for producing polypropylene which is a polymer used in a wide variety of applications. This process is complicated and the efficiency of the process depends on many factors of production such as feed condition factor, process factor, and catalyst factor. Hence, the improvement of the process will result in higher productivity and less production waste. Suitable condition adjustment of the process is one of the methods to make the process more efficient. However, condition adjustment cannot be easily performed due to a large number of factors. In addition, each condition adjustment can cause instability in process performance and cause a lot of production waste. Therefore, finding the suitable condition before adjustment is essential.

Examples of the prior arts related to improvements of the method and system for optimizing alkane dehydrogenation process are as follows.

U.S. Pat. No. 11,264,121 B2 discloses a system for predicting real-time production of a chemical product in a plant-based on a subset among a set of parameters each monitored by one of a corresponding set of sensors at one of a corresponding set of measurement frequencies. The system comprises a memory, a communication interface, circuitry in communication with the memory and the communication interface the circuitry configured to acquire multiple series of timestamped historical sensor data, obtain a series of timestamped and indirectly measured historical production data, sample the multiple series of timestamped historical sensor data of the set of parameters, interpolate the series of historical production data based on a local smoothing algorithm, filter the series of modified production data, separately apply a first dimensionality reduction algorithm and a second dimensionality reduction algorithm on the multiple series of sampled historical sensor data, and select the subset of parameters, develop a predictive model for production of the chemical product, store the predictive model in the memory, obtain real-time readings during production of the chemical product from a subset of sensors, and predict production of the chemical product based on the predictive model and the real-time readings of the subset of parameters from the subset of sensors.

U.S. Pat. No. 10,953,377 B2 discloses a delta temperature controller may determine and control the differential temperature across the reactor and use a delta temperature to control a set point for a heater temperature controller. By doing so, the plant may ramp up a catalytic dehydrogenation unit faster and ensure it does not coke up the catalyst and/or foul screens too quickly. Catalyst activity may be taken into account and allow the plant to have better control over the production and run length of the unit.

US 2020/0108327 A1 discloses methods, systems, and apparatuses for modifying plant operating conditions for the production of a product based on composition measurements associated with a distillation column. A control device may receive one or more composition measurements from a composition measurement device. The measurements may be associated with a distillation column of the plant. Based on the measurements, the control device may determine control instructions, e.g., using a history of control instructions. The plant may, based on the control decisions, interpret, and implement the instructions.

However, the method and system for optimizing an alkane dehydrogenation operation according to the above-mentioned patent documents still have some limitations. For example, those methods and systems still have high errors. Specifically, those patent documents do not disclose the prediction of the coking rate and do not disclose the optimization of the process in an account of the effects of both the product yield and the coking rate.

### SUMMARY OF THE INVENTION

An objective of the present invention is to provide an improved method and system for predicting the product yield and the coking rate in an accurate manner and at least 60 days in advance.

Another objective of the present invention is to provide the method and system capable of optimizing the production parameter data of the process in an account of the effects of both the product yield and the coking rate.

To achieve the aforementioned objectives, in a first aspect, this invention provides a method for optimizing an alkane dehydrogenation operation comprising the steps of:
(i) obtaining historical data of a plurality of production parameter data comprising feed condition parameter data, process parameter data, and catalyst parameter data from a production data detecting and storage unit;
(ii) filtering abnormal data of the historical data of the production parameter data;
(iii) developing a product yield prediction model and a coking rate prediction model by using at least some of the filtered historical data of the production parameter data; and
(iv) processing the product yield prediction model and the coking rate prediction model by using a process efficiency analysis model with a machine learning processing unit for predicting a product yield and a coking rate, and determining optimum production parameter data based on the predicted product yield and the predicted coking rate.

In a preferred embodiment, the step (iv) comprises sending real-time data of the production parameter data into the process efficiency analysis model; adjusting an accuracy of the real-time data of the production parameter data using a mass balance method and a heat balance method; predicting the product yield and the coking rate by using the adjusted real-time data of the production parameter data in the product yield prediction model and the coking rate prediction model, respectively; optimizing the used real-time data of the production parameter data in both of the product yield prediction model and the coking rate prediction model with the machine learning processing unit such that the product yield is controlled to be equal to or higher than a predetermined level and the coking rate is restricted from exceeding the predetermined level; and selecting the optimized process parameter data such that the product yield is highest and the coking rate does not exceed the predetermined level.

In a second aspect, the inventor of the present invention developed a system for optimizing an alkane dehydrogenation operation comprising:
a production data detecting and storage unit that detects and stores a plurality of production parameter data comprising feed condition parameter data, process parameter data, and catalyst parameter data, and
a processor configured to obtain historical data of the production parameter data from the production data detecting and storage unit; filter abnormal data of the historical data of the production parameter data; develop a product yield prediction model and a coking rate prediction model by using at least some of the filtered historical data of the production parameter data; and provide a process efficiency analysis model with a machine learning processing unit for processing the product yield prediction model and the coking rate prediction model to predict a product yield and a coking rate, and determine optimum production parameter data based on the predicted product yield and the predicted coking rate.

According to the second aspect of this invention, the process efficiency analysis model with the machine learning processing unit is configured to:
obtain current data of the production parameter;
adjust an accuracy of the current data of the production parameter data using a mass balance method and a heat balance method;
predict the product yield and the coking rate by using the adjusted real-time data of the production parameter data in the product yield prediction model and the coking rate prediction model, respectively;
optimize the used real-time data of the production parameter data in both of the product yield prediction model and the coking rate prediction model with the machine learning processing unit such that the product yield is controlled to be equal to or higher than a predetermined level and the coking rate is restricted from exceeding the predetermined level; and
select the optimized process parameter data such that the product yield is highest and the coking rate does not exceed the predetermined level.

### BRIEF DESCRIPTION OF THE DRAWINGS

FIG. 1 is a schematic diagram showing a procedure of optimizing an alkane dehydrogenation operation according to an exemplary method of the present invention.

FIG. 2 is a schematic diagram showing a procedure of processing the product yield prediction model and the coking rate prediction model according to an exemplary method of the present invention.

FIG. 3 is another schematic diagram showing a procedure of optimizing an alkane dehydrogenation operation according to an exemplary method of the present invention.

FIG. 4 is a diagram showing the components of the system according to the present invention.

FIG. 5 is graphs showing an optimization of the propane dehydrogenation operation using the method and system according to the present invention compared to those of the conventional optimization method and system.

### DETAILED DESCRIPTION

Any aspects shown herein shall encompass the application to other aspects of the present invention as well, unless specified otherwise.

Technical terms and scientific terms used herein have the definitions as understood by a person of ordinary skill in the art, unless specified otherwise.

The terms “consist(s) of,” “comprise(s),” “contain(s),” and “include(s)” are open-end verbs. For example, any method which “consists of,” “comprises,” “contains” or “includes” one component or multiple components or one step or multiple steps is not limited to only one component or one step or multiple steps or multiple components as specified but shall encompass components or steps that are not specified.

Any tools, devices, methods, materials, or calculation formulas mentioned herein, unless specified otherwise, mean the tools, devices, methods, materials, or calculation formulas generally used or practiced by a person skilled in the art.

The details of the invention will now be described in conjunction with the accompanying drawings for a better understanding.

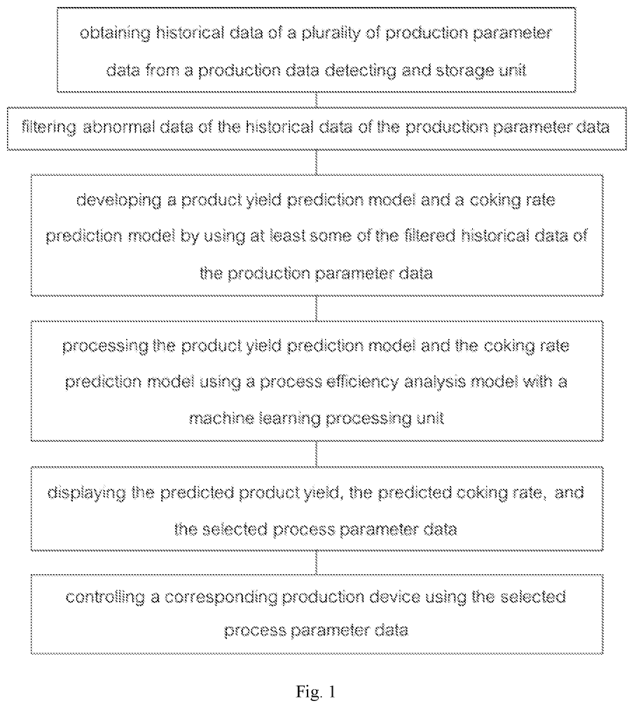

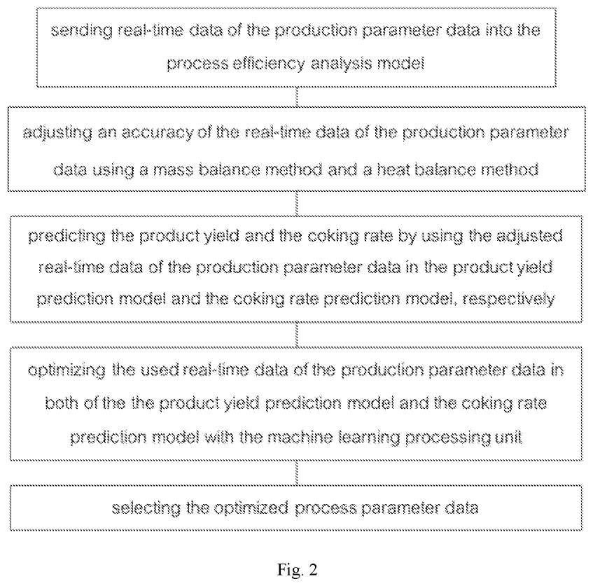

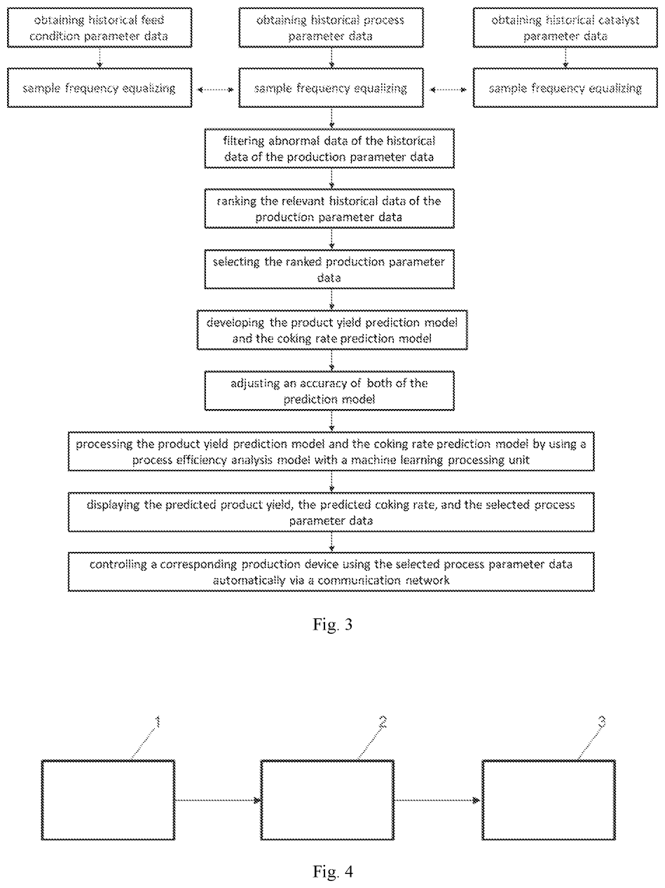

As shown in FIGS. 1-3, in the first aspect of invention, the method for optimizing an alkane dehydrogenation operation according to the present invention comprises the steps of:
(i) obtaining historical data of a plurality of production parameter data comprising feed condition parameter data, process parameter data, and catalyst parameter data from a production data detecting and storage unit;
(ii) filtering abnormal data of the historical data of the production parameter data;
(iii) developing a product yield prediction model and a coking rate prediction model by using at least some of the filtered historical data of the production parameter data; and
(iv) processing the product yield prediction model and the coking rate prediction model by using a process efficiency analysis model with a machine learning processing unit for predicting a product yield and a coking rate, and determining optimum production parameter data based on the predicted product yield and the predicted coking rate.

According to a preferred embodiment, the step (iv) comprises:
sending real-time data of the production parameter data into the process efficiency analysis model;
adjusting an accuracy of the real-time data of the production parameter data using a mass balance method and a heat balance method;
predicting the product yield and the coking rate by using the adjusted real-time data of the production parameter data in the product yield prediction model and the coking rate prediction model, respectively;
optimizing the used real-time data of the production parameter data in both of the product yield prediction model and the coking rate prediction model with the machine learning processing unit such that the product yield is controlled to be equal to or higher than a predetermined level and the coking rate is restricted from exceeding the predetermined level; and
selecting the optimized process parameter data such that the product yield is highest, and the coking rate does not exceed the predetermined level.

According to the above embodiment, providing the coking rate prediction model for predicting the coking rate allows an operator to recognize the status of the coking rate of the dehydrogenation process, in particular at least 60 days in advance, thereby the operator can improve the production process before the problems arise. For examples, the problems that the dehydrogenation process stopped by the coking rate being too high or high coking rate causing a short run-length of the process can be monitored and avoid.

Sending real-time data of the production parameter data into the process efficiency analysis model gives the advantage that the accuracy of the product yield prediction model and the coking rate prediction model to be rechecked by comparison with the actual product yield.

The adjustment of an accuracy of the real-time data of the production parameter data using a mass balance method and a heat balance method results in more accuracy for prediction.

Using the machine learning processing unit in the model according to the present invention results in more efficient and faster prediction.

Optimizing the used real-time data of the production parameter data in both the product yield prediction model and the coking rate prediction model can improve the current operating process and prevent short run-length of the operating process in the future.

According to the present invention, the abnormal data in the filtering step is data having a distribution outside a predetermined distribution range. Filtering the data in the above fashion allows historical data of a plurality of production parameter data to be corrected and a better representation. Examples of such data include the collected data at the time of the machine's malfunction or the time of the sensor's malfunction or human error in manual estimation and recording of the production parameter data.

For an embodiment of this invention, the feed condition parameter data, the process parameter data, and the catalyst parameter data are used to define a relation for developing the product yield prediction model and the coking rate prediction model. However, it is not necessary to use all those data. The product yield prediction model may opt to use any one of the feed condition parameter data, the process parameter data, and the catalyst parameter data. Similarly, the coking rate prediction model may opt to use any one of the feed condition parameter data, the process parameter data.

In more specific embodiment of this invention, developing a product yield prediction model and a coking rate prediction model comprises the steps of:
ranking the relevant historical data of the production parameter data using Pearson correlation method;
selecting the ranked production parameter data using Pearson correlation coefficient;
developing the product yield prediction model and the coking rate prediction model with the selected production parameter data using a linear regression method by the machine learning processing unit; and
adjusting an accuracy of the product yield prediction model and the coking rate prediction model such that a prediction error value does not exceed a predetermined value, for example, the predetermined value of the prediction error value being less than 5%.

According to this invention, the above steps are primarily performed to prioritize data based on magnitude and direction for use in selecting data and then to be tested and corrected to obtain the model having the predetermined prediction error value.

Preferably, the product yield prediction model and the coking rate prediction model are based on a general linear equation.

From above, using the general linear equation has advantages that the equation is simple and less complicated to execute which can be applied on many platforms with minimum implementation cost.

Examples of the feed condition parameter data can be those selected from at least one of a hydrogen concentration, a hydrocarbon concentration, a hydrogen to hydrocarbon ratio, and a flow rate of an anti-coking catalyst and corrosion inhibitor.

Examples of the process parameter data can be those selected from at least one of a reactor inlet temperature, a fuel gas pressure, a percentage of oxygen excess in radiation zone of heater, total combined feed rate, and a lift gas velocity.

Examples of the catalyst parameter data can be those selected from at least one of a catalyst circulation rate, a volume of fine catalyst, an accumulation of fine catalyst, a top-up volume of catalyst, a catalyst regeneration temperature and a flow rate of a catalyst dispersing agent.

The parameter selection is not limited to those specified and the choice of data may be increased as appropriate.

The optimization method according to the present invention may further comprise a step of equalizing a sample frequency of each historical data of the feed condition parameter data, the process parameter data, and the catalyst parameter data prior to filtering the abnormal data, in case that the sample frequency of each historical data is inconsistent, thereby such equalizing the sample frequency of each historical data of the feed condition parameter data, the process parameter data, and the catalyst parameter data is processed using the said data that is last updated prior to the same period.

Equalizing the sample frequency has the advantage that data will conform and represent actual condition of the process which make model have more accuracy.

Alternatively, the optimization method may further comprise displaying the predicted product yield, the predicted coking rate, and the selected process parameter data to a user.

Another alternative embodiment, the optimization method may further comprise controlling a corresponding production device using the selected process parameter data automatically via a communication network. Examples of the corresponding production device may include process control devices such as flow control valves, heaters, temperature control valves, pressure control valves, etc.

The second aspect of the present invention relates to the system for optimizing an alkane dehydrogenation operation. As shown in FIG. 4, the system of the present invention comprises a production data detecting and storage unit (1) that detects and stores a plurality of production parameter data comprising feed condition parameter data, process parameter data, and catalyst parameter data; and a processor (2) configured to:
obtain historical data of the production parameter data from the production data detecting and storage unit (1);
filter abnormal data of the historical data of the production parameter data;
develop a product yield prediction model and a coking rate prediction model by using at least some of the filtered historical data of the production parameter data; and
provide a process efficiency analysis model with a machine learning processing unit for processing the product yield prediction model and the coking rate prediction model to predict a product yield and a coking rate, and determine optimum production parameter data based on the predicted product yield and the predicted coking rate.

According to an exemplary embodiment, the production data detecting and storage unit (1) may include laboratory information system, laboratory management system, plant information system, manual estimation and recording of the production parameter data, etc.

According to the present invention, the process efficiency analysis model with the machine learning processing unit is configured to:
obtain current data of the production parameter;
adjust an accuracy of the current data of the production parameter data using a mass balance method and a heat balance method;
predict the product yield and the coking rate by using the adjusted real-time data of the production parameter data in the product yield prediction model and the coking rate prediction model, respectively;
optimize the used real-time data of the production parameter data in both of the product yield prediction model and the coking rate prediction model with the machine learning processing unit such that the product yield is controlled to be equal to or higher than a predetermined level and the coking rate is restricted from exceeding the predetermined level; and
select the optimized process parameter data such that the product yield is highest and the coking rate does not exceed the predetermined level.

In a preferred embodiment, the abnormal data is data having a distribution being outside a predetermined distribution range.

According to the present invention, the feed condition parameter data, the process parameter data, and the catalyst parameter data are used to define a relation for developing the product yield prediction model, and the coking rate prediction model.

The processor (2) of this invention is configured to use at least some of the filtered historical data of the production parameter data to develop the product yield prediction model, and the coking rate prediction model is configured to:
rank the relevant historical data of the production parameter data based on magnitude and direction using Pearson correlation method;
select the ranked production parameter data using Pearson correlation coefficient;
develop the product yield prediction model and the coking rate prediction model with the selected production parameter data using a linear regression method by the machine learning processing unit; and
adjust an accuracy of the product yield prediction model and the coking rate prediction model such that a prediction error value does not exceed a predetermined value.

Preferably, the predetermined value of the prediction error value is less than 5%.

The product yield prediction model and the coking rate prediction model are based on a general linear equation.

The feed condition parameter data may be selected from at least one of a hydrogen concentration, a hydrocarbon concentration, a hydrogen to hydrocarbon ratio, and a flow rate of an anti-coking catalyst and corrosion inhibitor.

The process parameter data may be selected from at least one of a reactor inlet temperature, a fuel gas pressure, a percentage of oxygen excess in radiation zone of heater, total combined feed rate, and a lift gas velocity.

The catalyst parameter data may be selected from at least one of a catalyst circulation rate, a volume of fine catalyst, an accumulation of fine catalyst, a top-up volume of catalyst, a catalyst regeneration temperature and a flow rate of a catalyst dispersing agent.

In more preferred embodiment, the processor (2) is configured to equalize a sample frequency of each historical data of the feed condition parameter data, the process parameter data, and the catalyst parameter data prior to filtering the abnormal data, in case that the sample frequency of each historical data is inconsistent. Preferably, the processor (2) equalizes the sample frequency of each historical data by using the said data that is last updated prior to the same period.

The system of the present invention may further comprise a display (3) connected to the processor (2) to receive and display the predicted product yield, the predicted coking rate, and the selected process parameter data to a user. For examples, the display (3) may include PC computer, laptop, mobile, tablet, etc.

The processor (2) according to the present invention may be configured to control a corresponding production device using the selected process parameter data automatically via a communication network. The control of corresponding production device by the processor (2) can reduce user's burden of manually adjusting the device.

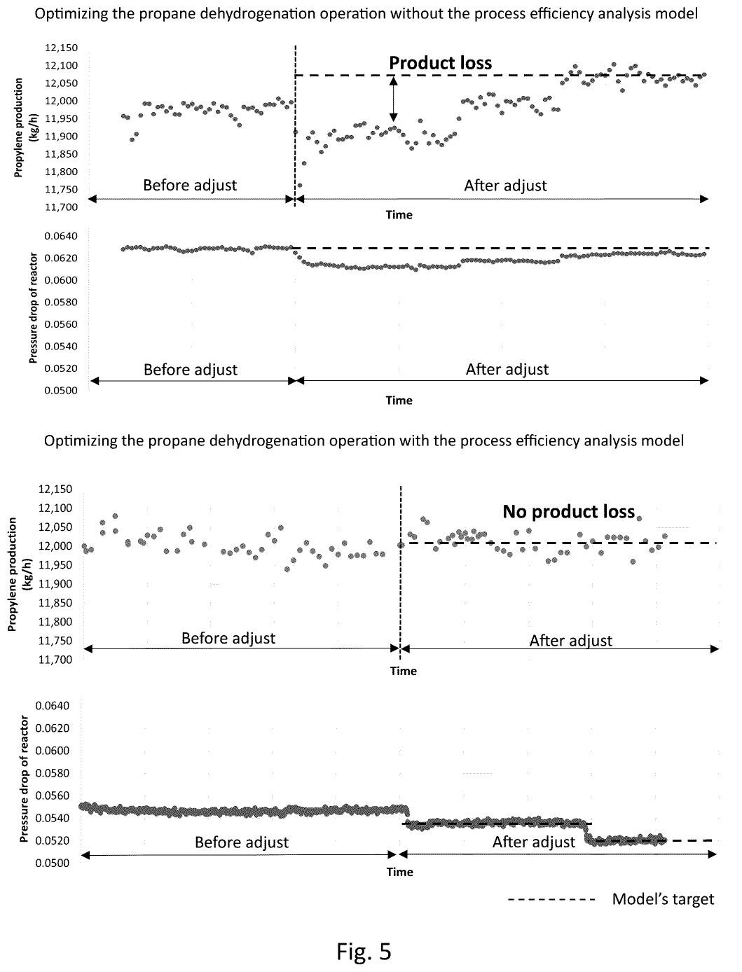

The inventor has conducted tests for optimizing the propane dehydrogenation operation using the method and system according to this invention compared to a conventional optimization method (e.g., Trial and Error method). The test results are shown in FIG. 5. It is found from the results that the propane dehydrogenation process which is adjusted with the method and system of the present invention does not cause product loss after the adjustment, while the propane dehydrogenation process which is adjusted with the conventional method and system causes significant product loss after the adjustment. Further, the coking rate (based on the pressure drop of the reactor which are lead indicator for plant shutdown) of the propane dehydrogenation process which is adjusted with the method and system of the present invention is significantly reduced and stable.

The implementation of the method of the present invention previously described which comprises various steps may be performed in any other order different from the one described. Any modifications and changes evident to a person of ordinary skilled in the art should be considered to be within the spirit, scope, and concept of the present invention.

1. A method for optimizing an alkane dehydrogenation operation, the method comprising the steps of:
(i) obtaining historical data of a plurality of production parameter data comprising feed condition parameter data, process parameter data, and catalyst parameter data from a production data detecting and storage unit;
(ii) filtering abnormal data of the historical data of the production parameter data;
(iii) developing a product yield prediction model and a coking rate prediction model by using at least some of the filtered historical data of the production parameter data; and
(iv) processing the product yield prediction model and the coking rate prediction model by using a process efficiency analysis model with a machine learning processing unit for predicting a product yield and a coking rate, and determining optimum production parameter data based on the predicted product yield and the predicted coking rate;
wherein the step (iv) comprises:
sending real-time data of the production parameter data into the process efficiency analysis model;
adjusting an accuracy of the real-time data of the production parameter data using a mass balance method and a heat balance method;
predicting the product yield and the coking rate by using the adjusted real-time data of the production parameter data in the product yield prediction model and the coking rate prediction model, respectively;
optimizing the used real-time data of the production parameter data in both of the product yield prediction model and the coking rate prediction model with the machine learning processing unit such that the product yield is controlled to be equal to or higher than a predetermined level and the coking rate is restricted from exceeding the predetermined level; and
selecting the optimized process parameter data such that the product yield is highest and the coking rate does not exceed the predetermined level.

2. The method of claim 1, wherein the abnormal data is data having a distribution being outside a predetermined distribution range.

3. The method of claim 1, wherein
the feed condition parameter data, the process parameter data, and the catalyst parameter data are used to define a relation for developing the product yield prediction model, and the coking rate prediction model.

4. The method of claim 1, wherein the step (iii) comprises the steps of:
ranking the relevant historical data of the production parameter data using Pearson correlation method;
selecting the ranked production parameter data using Pearson correlation coefficient;
developing the product yield prediction model and the coking rate prediction model with the selected production parameter data using a linear regression method by the machine learning processing unit; and
adjusting an accuracy of the product yield prediction model and the coking rate prediction model such that a prediction error value does not exceed a predetermined value.

5. The method of claim 4, wherein the predetermined value of the prediction error value is less than 5%.

6. The method of claim 1, wherein the product yield prediction model and the coking rate prediction model are based on a general linear equation.

7. The method of claim 1, wherein the feed condition parameter data is selected from at least one of a hydrogen concentration, a hydrocarbon concentration, a hydrogen to hydrocarbon ratio, and a flow rate of an anti-coking catalyst and corrosion inhibitor.

8. The method of claim 1, wherein the process parameter data is selected from at least one of a reactor inlet temperature, a fuel gas pressure, a percentage of oxygen excess in radiation zone of heater, total combined feed rate, and a lift gas velocity.

9. The method of claim 1, wherein the catalyst parameter data is selected from at least one of a catalyst circulation rate, a volume of fine catalyst, an accumulation of fine catalyst, a top-up volume of catalyst, a catalyst regeneration temperature and a flow rate of a catalyst dispersing agent.

10. The method of claim 1 further comprising equalizing a sample frequency of each historical data of the feed condition parameter data, the process parameter data, and the catalyst parameter data prior to filtering the abnormal data, in case that the sample frequency of each historical data is inconsistent.

11. The method of claim 10, wherein equalizing the sample frequency of each historical data of the feed condition parameter data, the process parameter data, and the catalyst parameter data is processed using the said data that is last updated prior to the same period.

12. The method of claim 1 further comprising displaying the predicted product yield, the predicted coking rate, and the selected process parameter data to a user.

13. The method of claim 1 further comprising controlling a corresponding production device using the selected process parameter data automatically via a communication network.

14. A system for optimizing an alkane dehydrogenation operation, comprising:
a production data detecting and storage unit (1) that detects and stores a plurality of production parameter data comprising feed condition parameter data, process parameter data, and catalyst parameter data; and
a processor (2) configured to:
obtain historical data of the production parameter data from the production data detecting and storage unit (1);
filter abnormal data of the historical data of the production parameter data;
develop a product yield prediction model and a coking rate prediction model by using at least some of the filtered historical data of the production parameter data; and
provide a process efficiency analysis model with a machine learning processing unit for processing the product yield prediction model and the coking rate prediction model to predict a product yield and a coking rate, and determine optimum production parameter data based on the predicted product yield and the predicted coking rate;
wherein the process efficiency analysis model with the machine learning processing unit is configured to:
obtain current data of the production parameter;
adjust an accuracy of the current data of the production parameter data using a mass balance method and a heat balance method;
predict the product yield and the coking rate by using the adjusted real-time data of the production parameter data in the product yield prediction model and the coking rate prediction model, respectively;
optimize the used real-time data of the production parameter data in both of the product yield prediction model and the coking rate prediction model with the machine learning processing unit such that the product yield is controlled to be equal to or higher than a predetermined level and the coking rate is restricted from exceeding the predetermined level; and
select the optimized process parameter data such that the product yield is highest and the coking rate does not exceed the predetermined level.

15. The system of claim 14, wherein the abnormal data is data having a distribution being outside a predetermined distribution range.

16. The system of claim 14, wherein the feed condition parameter data, the process parameter data, and the catalyst parameter data are used to define a relation for developing the product yield prediction model, and the coking rate prediction model.

17. The system of claim 14, wherein
the processor (2) is configured to use at least some of the filtered historical data of the production parameter data to develop the product yield prediction model, and the coking rate prediction model is configured to:
rank the relevant historical data of the production parameter data based on magnitude and direction using Pearson correlation method;
select the ranked production parameter data using Pearson correlation coefficient;
develop the product yield prediction model and the coking rate prediction model with the selected production parameter data using a linear regression method by the machine learning processing unit; and
adjust an accuracy of the product yield prediction model and the coking rate prediction model such that a prediction error value does not exceed a predetermined value.

18. The system of claim 17, wherein the predetermined value of the prediction error value is less than 5%.

19. The system of claim 14, wherein the product yield prediction model and the coking rate prediction model are based on a general linear equation.

20. The system of claim 14, wherein the feed condition parameter data is selected from at least one of a hydrogen concentration, a hydrocarbon concentration, a hydrogen to hydrocarbon ratio, and a flow rate of an anti-coking catalyst and corrosion inhibitor.

21. The system of claim 14, wherein the process parameter data is selected from at least one of a reactor inlet temperature, a fuel gas pressure, a percentage of oxygen excess in radiation zone of heater, total combined feed rate, and a lift gas velocity.

22. The system of claim 14, wherein the catalyst parameter data is selected from at least one of a catalyst circulation rate, a volume of fine catalyst, an accumulation of fine catalyst, a top-up volume of catalyst, a catalyst regeneration temperature and a flow rate of a catalyst dispersing agent.

23. The system of claim 14, wherein the processor (2) is configured to equalize a sample frequency of each historical data of the feed condition parameter data, the process parameter data, and the catalyst parameter data prior to filtering the abnormal data, in case that the sample frequency of each historical data is inconsistent.

24. The system of claim 23, wherein the processor (2) equalizes the sample frequency of each historical data by using the said data that is last updated prior to the same period.

25. The system of claim 14 further comprising a display (3) connected to the processor (2) to receive and display the predicted product yield, the predicted coking rate, and the selected process parameter data to a user.

26. The system of claim 14, wherein the processor (2) is configured to control a corresponding production device using the selected process parameter data automatically via a communication network.
